(19) United States
(12) Patent Application Publication
(10) Pub. No.: US 2025/0120625 A1
(43) Pub. Date: Apr. 17, 2025

(54) MOLECULARLY IMPRINTED WEARABLE SENSOR WITH PAPER MICROFLUIDICS FOR REAL TIME SWEAT BIOMARKER ANALYSIS

(71) Applicant: The Texas A&M University System, College Station, TX (US)

(72) Inventors: Limei Tian, College Station, TX (US); Mayank Garg, College Station, TX (US)

(73) Assignee: The Texas A&M University System, College Station, TX (US)

(21) Appl. No.: 18/914,168

(22) Filed: Oct. 12, 2024

Related U.S. Application Data
(60) Provisional application No. 63/543,929, filed on Oct. 12, 2023.

(51) Int. Cl. A61B 5/1477; A61B 5/00; A61B 5/145

(52) U.S. Cl. CPC A61B 5/1477; A61B 5/14514; A61B 5/14521; A61B 5/14539; A61B 5/14546; A61B 5/4266; A61B 5/6832; A61B 2560/0468; A61B 2562/0215; A61B 2562/16; A61B 2562/18

(57) ABSTRACT

A wearable biosensor for real-time quantification of a biomarker in a biofluid includes at least one functional module. The functional module includes an iontophoresis induction module, a microfluidic layer, a plurality of multimodal biosensors, and a paperfluidic layer.

### CROSS-REFERENCE TO RELATED APPLICATIONS

This patent application claims priority from, and incorporates by reference the entire disclosure of, U.S. Provisional Patent Application No. 63/543,929 filed on Oct. 12, 2023.

### STATEMENT REGARDING FEDERALLY SPONSORED RESEARCH

This invention was made with government support under 1R35GM147568-01 awarded by the National Institutes of Health. The government has certain rights in the invention.

### TECHNICAL FIELD

The present disclosure relates generally to biosensors and more particularly, but not by way of limitation, to wearable biosensors for real-time quantification of biomarkers in sweat.

### BACKGROUND

Wearable devices are a key step towards achieving the goal of personalized health monitoring. The ability of detecting minute concentrations of biomarkers with extreme precision is of utmost importance and at the same time, highly challenging. Detection of cortisol is of prime importance in order to estimate stress levels of a subject. The reported methods for the detection of cortisol involve the use of antibodies, aptamers or molecular imprinted polymers modified electrodes with the signal recording being done in the presence of a redox probe. The use of a redox probe not only limits the application but also makes the sensor less reliant due to toxicity of the redox probe.

The rapid, sensitive, and accurate determination of biomarkers in sweat remains a clinical challenge. Sweat is a highly sought after biofluid for wearable sensors since it reflects the nutritional and biochemical changes taking place in the body. These biochemical changes can be tracked via wearable sensors in a non-invasive, continuous, and real-time fashion. Since the secreted sweat volume is low, highly sensitive and sophisticated microfluidic devices (e.g., devices that incorporate microchannels) have been developed for the sweat collection and handling. These microfluidic devices have been coupled with sweat ionic sensors (e.g., sodium, chloride, and hydrogen ions) for the determination of the ions with changing sweat rate. Among the electrochemical means of sweat rate determination, determining the admittance as a function of time has been reported. Sweat rate sensing has also been achieved using colorimetric method. For the continuous sweat rate quantification, the sensing chamber needs to be reset after it becomes full. The fabrication of microchannels is challenging and complex. Hence, a simple and convenient sweat handling and analysis system, that can be replaced by users as well, must be developed.

Cortisol is a prominent stress hormone that is found in sweat and is a representation of the body stress levels. Since the concentration of this molecule is in the nanomolar range (22 to 390 nM), the sensors for the detection thereof need to be ultra-specific and highly sensitive. Transducer surfaces in some devices have been modified with antibodies, aptamers or with Molecularly Imprinted Polymers (MIPs). The electro-inactive nature of cortisol has made researchers employ different strategies for the signal generation. The use of anti-cortisol monoclonal antibodies modified laser induced graphene has been reported for cortisol detection in a wearable format. A competitive assay between the cortisol in sweat and an enzyme labelled cortisol was used, and a signal was generated through complex enzymatic reactions that involved the oxidation and reduction of horse radish peroxidase (HRP), hydroquinone and hydrogen peroxide. Though the method is sensitive, the use of enzymes and external redox mediators make the sensing process cumbersome. Others have demonstrated the use of a field-effect transistor (FET) device modified with cortisol specific aptamers for cortisol estimation in a wearable format. The aptamers in this device were immobilized to an Indium oxide FET device. This device was able to detect cortisol (1 pM to 1 μM) in artificial sweat samples. A complex biological fluid, such as sweat, can cause the conformational change or mis-folding of the aptamers making them not suitable candidates as recognition elements for a wearable sensor. MIPs present a unique advantage compared to the biological counterparts in terms of physiological stability, ease of fabrication and high specificity. While many MIP based wearable cortisol sensors have been reported, they rely on the use of a redox active molecule or the readout involves a redox active molecule. Though the sensor construction and readout simplify this method somewhat, the use of redox active molecule is an additional chemical that adds complexity.

The design of the electrode is a major factor in the development of cost-effective and scalable wearable devices. Lithography-based gold electrodes allow for the fabrication of minute features on the electrode surface, but the method overall becomes very expensive. On the other hand, production of laser engraving based graphene electrodes is scalable, cost-effective, and efficient. The graphene produced during the process is more electroactive than the gold electrodes due to the high surface area and porosity. The laser engraved graphene-based MIP sensors however, also rely on a redox probe for signal generation, wherein cortisol (outside physiological concentration range) is detected along with many metabolites using MIP modified LEG sensor.

In order to maximize the information that can be extracted from a given amount of sweat, research has been done showing integrated sensors detecting multiple biomarkers in sweat at a given point of time. The previous works have given primary importance to detection of glucose and alcohol in the sweat.

### SUMMARY OF THE INVENTION

This summary is provided to introduce a selection of concepts that are further described below in the Detailed Description. This summary is not intended to identify key or essential features of the claimed subject matter, nor is it to be used as an aid in limiting the scope of the claimed subject matter.

According to an exemplary aspect, a wearable biosensor for real-time quantification of a biomarker in a biofluid includes at least one functional module. The at least one functional module includes an iontophoresis induction module, a microfluidic layer, a plurality of multimodal biosensors, and a paperfluidic layer. In some aspects, the biofluid is sweat or interstitial fluid.

In some aspects, the iontophoresis induction module comprises two LIG electrodes coated with hydrogels. In some aspects, LIG electrodes are fabricated on polyimide sheet.

In some aspects, the two LIG electrodes are an anode and a cathode. In some aspects, the anode electrode comprises a carbachol-loaded hydrogel coating and is positioned to capture the biofluid. In some aspects, the cathode electrode comprises a sodium chloride hydrogel coating and is configured to complete an electrical circuit.

In some aspects, the microfluidic layer comprises an inlet layer.

In some aspects, the inlet layer is made of double-sided skin adhesive that is configured to form a mechanically robust interface between the skin of a user and the wearable biosensor. In some aspects, the inlet layer comprise two arc shaped openings to allow direct contact between the hydrogel and the skin of a user. In some aspects, the inlet layer comprises one circular opening fluidly coupled to the paperfluidic layer for biofluid volume and rate quantification. In some aspects, the inlet layer has three circular openings to collect the biofluid for quantifying pH, sodium ion (Na+) and cortisol concentrations.

In some aspects, the wearable biosensor includes a paper wicking layer that connects to the paperfluidic layer of the wearable biosensor to efficiently remove accumulated biofluid after quantification is completed. In some aspects, wherein the paper wicking layer comprises chromatography paper.

In some aspects, the wearable biosensor is encapsulated with a skin adhesive to minimize biofluid evaporation and to protect the device from environmental contamination.

In some aspects, the measured biomarker is cortisol.

In some aspects, the wearable biosensor includes a first LIG electrode configured as a counter electrode, a second LIG electrode comprising an Ag/AgCl coating, and a pair of working electrodes. In some aspects, a working electrode of the pair of working electrodes is an LIG electrode comprising an electrochemically synthesized cortisol-specific Molecularly Imprinted Polymer coating for cortisol detection and quantification. In some aspects, a working electrode of the pair of working electrodes is an LIG electrode comprising a polypyrrole and sodium ion-selective membrane to quantify sweat Na+ concnetration.

### BRIEF DESCRIPTION OF THE DRAWINGS

A more complete understanding of the subject matter of the present disclosure may be obtained by reference to the following Detailed Description when taken in conjunction with the accompanying Drawings wherein:

FIG. 1A is an exploded assembly illustrating a flexible, wearable device for analyzing a biofluid, according to aspects of the disclosure;

FIG. 1B is a schematic representation of LIG electrodes exposed to a biofluid, according to aspects of the disclosure;

FIGS. 1C and 1D are front and back views, respectively, of the flexible, wearable device of FIG. 1;

FIG. 2 illustrates the flexible, wearable device placed on an arm of a user, according to aspects of the disclosure; and

FIGS. 3A-3C three different configurations of LIG electrodes employing different dimensions and geometries, according to aspects of the disclosure.

### DESCRIPTION

A flexible, wearable device that can induce biofluid via iontophoresis on demand and simultaneously quantify several parameters, including volume, secretion rate, sodium ion, and cortisol, hormone, dug, metabolites, proteins, pathogens, and other concentrations is discussed herein. Biofluid is used herein to include interstitial fluids, including sweat. The devices and methods discussed herein may be described relative to sweat, but it will be understood that the devices and methods may be used more generally with biofluids. The device includes integrated paper microfluidic modules that enable the quantification of sweat volume and secretion rate, and also function as a pump to remove the collected sweat from the sensing chamber once the chamber is filled. This reset approach does not involve user intervention and eliminates the mixing of sweat collected at different times. MIP was electrochemically synthesized on laser-induced graphene (LIG) electrodes, which enables cortisol binding to occur within the Debye length for high sensitivity. The MIP-based sensor detects and quantifies the cortisol level in sweat without the presence of labels and redox probes using electrochemical impedance spectroscopy (EIS). The ionic selective membrane-coated electrode was used to quantify sodium ion concentration through open circuit potential measurements. The wearable device can be used to measure these sweat parameters on a human subject.

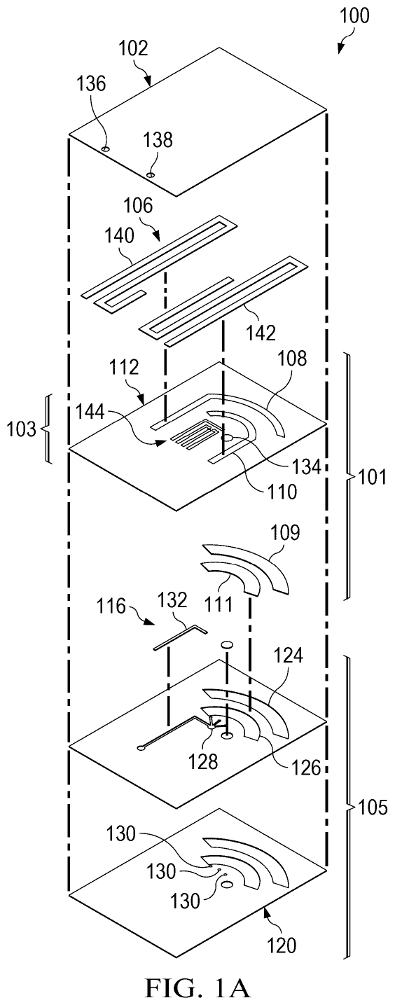

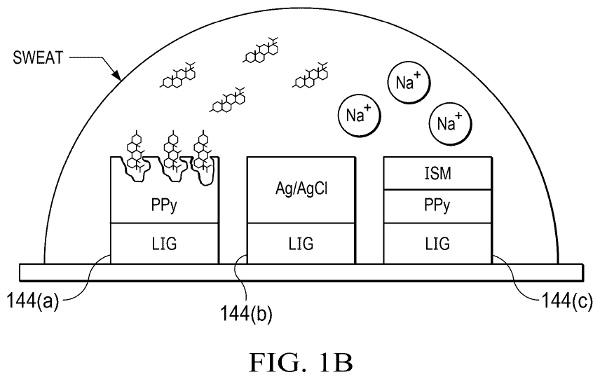

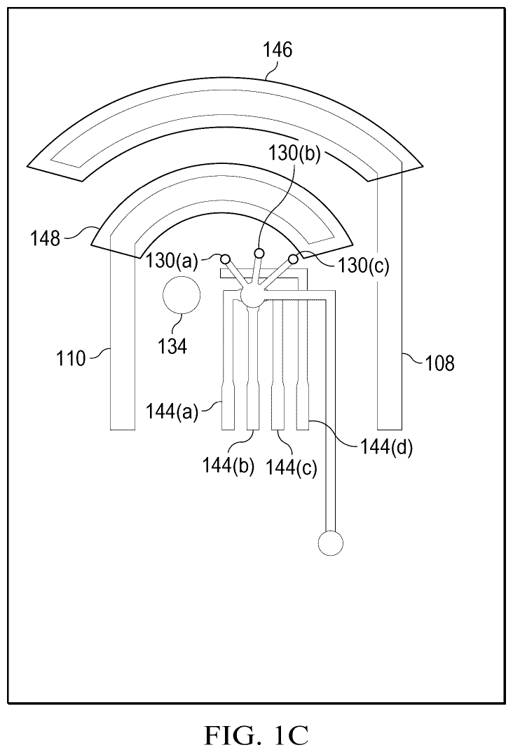

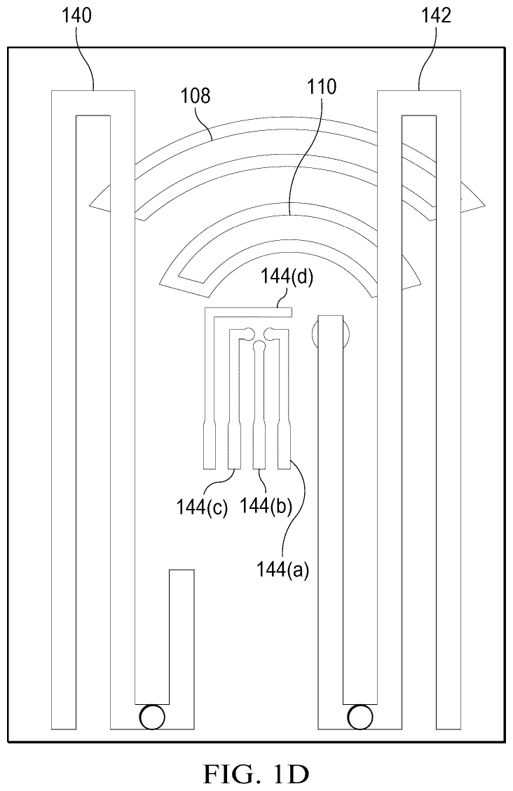

FIG. 1A illustrates a flexible, wearable device 100 that comprises several functional modules, including an iontophoresis sweat induction module 101, multimodal biosensors 103, a microfluidic chamber 105, and a paper microfluidic layer 106. Iontophoresis sweat induction module 101 includes two arc-shaped graphene electrodes 108, 110 coated with hydrogels 109, 111 to stimulate sweat secretion on demand. The anode electrode 110 is coated with a carbachol-loaded hydrogel layer and is configured to be in line with a microfluidic inlet of an inlet layer 120, where the secreted sweat is captured. The cathode electrode 108 is coated with sodium chloride hydrogel and completes the electrical circuit.

Microfluidic chamber 105 includes microchannels that transport sweat from inlet 130 to a sensing chamber 128 and then to the paper microfluidic (simplified as paperfluidic) layer 132. Sensing chamber 128 and inlet 130 are defined by a double-sided skin adhesive, forming a mechanically robust interface between the skin and the wearable device. Inlet 130 is comprised of three circular openings with a 1 mm diameter to collect sweat and transport it to the sensors for quantifying sodium ion (Na+) and cortisol concentrations. Paperfluidic layer 132 (0.5 mm wide and 2.2 cm long) serves as a pump and connects an outlet of sensing chamber 128 to a paperfluidic layer 140 on a backside of device 100 to efficiently remove accumulated sweat. Another inlet 134 has one circular opening with a 2 mm diameter opening connected to paperfluidic layer 106 for sweat volume and rate quantification. Inlet 134 is formed from several via holes formed between inlet layer 120 through an LIG layer 112 and is filled with wicking paper to facilitate sweat transportation. Finally, device 100 includes an outer or encapsulation layer 102 that sandwiches the layers of the device together between itself and the skin of the user. Encapsulation is used herein to denote that the layers of the device are sandwiched between encapsulation layer 102 and the skin of a user to form a protective coating or film. Encapsulation layer 102 may be a thin Tegaderm transparent film to minimize sweat evaporation and protect it from environmental contamination. The encapsulation layer 102 includes two outlets 136, 138 that align with ends of paperfluidics 140, 142 of paperfluidic layer 106 to eliminate back pressure that can impede sweat flow.

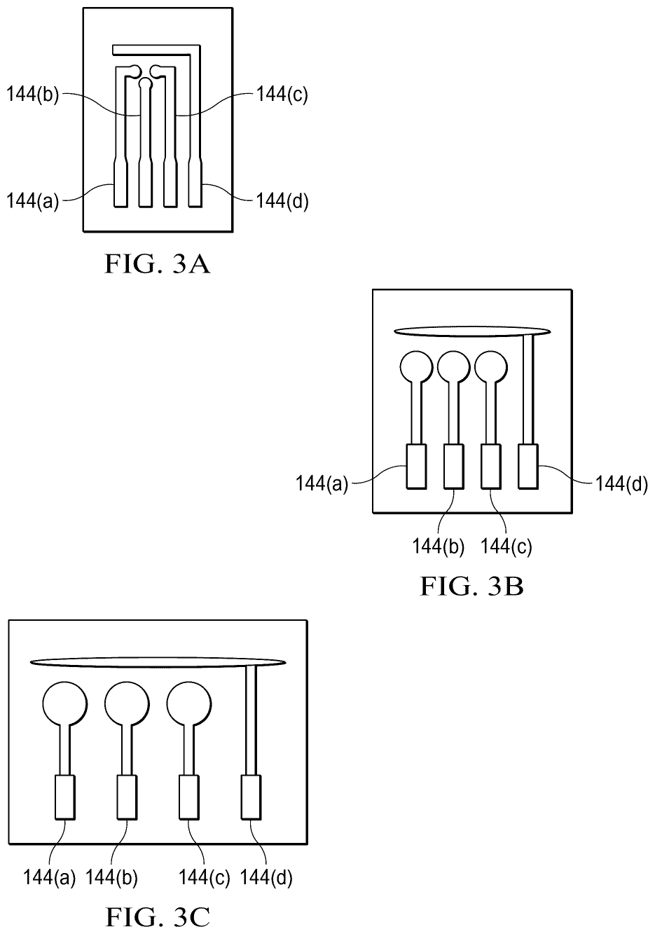

Laser scribing was used to fabricate porous LIG electrodes 144 (see also FIGS. 1C, 1D, and 3A-3C) on a flexible polyimide substrate, including sweat induction and sensing electrodes. This fabrication approach is simple, inexpensive, scalable, and enables mask-free graphene patterning on various substrates. FIGS. 1C and 1D are front and rear views of device 100 showing electrochemical sensors comprised of four electrodes 144(a)-144(d), including one LIG electrode as a counter electrode (144(d)), one LIG coated with Ag/AgCl as a reference electrode (144(b)), and two working electrodes (144(a), 144(c)) (see FIGS. 1B, 1C, 1D). One working electrode (144(a)) is LIG with electrochemically synthesized cortisol-specific MIP for cortisol detection and quantification. The other working electrode (144(c)) is LIG coated with polypyrrole and sodium ion-selective membrane to quantify sweat Na+ concentration. FIGS. 1C and 1D illustrate a front and back, respectively, of device 100 with all layers assembled. FIG. 1C shows the side interfaced with the skin. Device 100 includes two arc-shaped openings 146, 148 that are coated with hydrogel layers, which are directly in contact with the skin, for sweat stimulation. FIG. 1C also shows inlet 134 connected to paperfluidic channel 142 and three small inlets 130(a)-130(c) for capturing and transporting the sweat to sensing chamber 128. FIG. 1D shows paperfluidic channels 140, 142, with paperfluidic channel 140 removing the accumulated sweat from the sensing chamber at a well-defined rate and paperfluidic channel 142 quantifying sweat volume and secretion rate. Chromatography paper was used to construct paperfluidic channels 140, 142 because it has a well-defined flow rate and absorption capacity.

### Microfluidic Characterization and Sweat Volume/Rate Quantification

The wetting properties of microfluidic device surfaces have a significant impact on fluid flow characteristics. The LIG on the polyimide substrate was treated with oxygen plasma to enhance the hydrophilicity of surfaces. The untreated surface had a contact angle of 109.3±3.8°, while the freshly plasma-treated and one-year-old treated surfaces showed contact angles of 7.5±0.4° and 6.5±0.3°, respectively. These results demonstrated that plasma treatment makes the LIG surface hydrophilic, and this hydrophilicity is maintained even after one year. Testing demonstrated that an aqueous blue dye solution introduced to the inlet of the device rapidly filled the sensing chamber in less than 30 seconds, and was removed by the paperfluidic pump effectively over time. After 60 seconds, additional water was introduced to the inlet, and the dye solution was completely removed in 8 minutes. The paperfluidic pump can continuously transport the dye solution through capillary force and remove accumulated fluid from the sensing chamber without any sign of backflow. This prevents the mixing of sweat samples generated sequentially, which facilitates the accurate quantification of analytes with varying concentrations. Without oxygen plasma treatment, the dye solution remained at the inlet and could not flow into the sensing chamber due to the poor wetting properties of untreated LIG and polyimide.

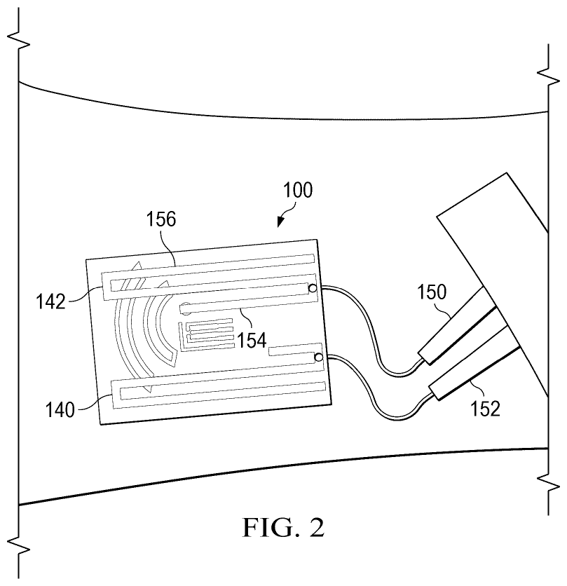

The liquid-wicking kinetics of paper microfluidics assembled on the polyimide and encapsulated by a thin Tegaderm transparent film were characterized and evaluated for their capability to quantify the sweat volume and rate in real time. The fluid flow rate in the paperfluidic channel was quantified by video recordings of fluid propagation after introducing an excess amount of fluid to the inlet. It was easy to visualize the fluid front on the paper substrate. The fluid traveled 106 mm along a 2 mm wide paperfluidic channel in 15 minutes. The fluid flow follows the Lucas-Washburn equation, which quantifies the correlation between travel distance, surface tension, viscosity of the liquid, and contact angle between the fluid and boundary wall and time. Although the paper wicking rate slows down over time, it remains higher than the typical human sweat rate of 12-120 μL/cm²·h. For comparison, the sweat rate induced by intense exercise was quantified and iontophoresis with the paperfluidic devices applied on the forearm of a healthy human subject. FIG. 2 illustrates device 100 secured to an arm of a user. FIG. 2 shows sweat being absorbed along paperfluidic channel 142 (e.g., sweat is shown dispersed throughout a first portion 154, working its way into a second portion 156). Device 100 is also shown connected to a pair of leads 150, 152. Optical images of the device collected at 15 minutes following intense cycling exercise showed the sweat travel distance of 86 mm. Optical images of the device were collected at 15 minutes following iontophoresis, which involves a small current of 100 μA applied through the sweat stimulation electrodes for 5 minutes. Compared to intense exercise, iontophoresis-induced sweat showed a shorter travel distance, suggesting a lower sweat volume. Comparison between the fluid travel distance upon wicking and sweat travel distance with iontophoresis and exercise over time showed that the travel distance linearly increased with increasing volume. The volume was derived using a controlled quantity of liquid to establish the relationship between the travel distance and volume of liquid absorbed by the paper microfluidic channel. The liquid volume and travel distance relationship was 4.74+0.16 mm/μL for the paper with a channel width of 2 mm. The sweat rates in both cases slowed down over time, but they were much slower than the paper wicking rate. These results confirmed the capability of the paperfluidic module for real-time sweat volume and rate quantification.

### LIG Electrode Characterization

With the laser scribing approach, it is easy to fabricate LIG electrodes and interconnects of different dimensions and geometries. FIGS. 3A-3C illustrate three different configurations of LIG electrodes 144(a)-144(d) employing different dimensions and geometries. Scanning electron microscope (SEM) imaging revealed a highly porous structure of graphene induced by laser scribing. LIG Raman spectra was collected with a Raman spectrometer at 514 nm, which showed three prominent peaks, including D, G, and 2D Raman peaks at ˜1350, 1580, and 2700 cm⁻¹, respectively. The D peak originates from the defect active breathing modes of six-atom rings. The G and 2D peaks correspond to the high-frequency E2g phonon and the second-order zone-boundary phonons, respectively. The 2D peak can be fitted with a single Lorentzian peak centered at ˜2,700 cm⁻¹, indicating the LIG is primarily comprised of single-layer graphene.

The electrochemical performance of a 3 mm-diameter LIG electrode was compared with a commercial screen-printed carbon (SPC) electrode of the same dimension. Cyclic voltammograms collected with two electrodes in K₄Fe(CN)₆/K₃Fe(CN)₆ as a reference system were created. The oxidation-reduction signature peaks of ferro-ferricyanide show that the LIG electrode has a higher current response than the SPC electrode. This can be attributed to the graphene's high surface area and electron mobility. Electrodes with changing diameters of 1, 2, and 3 mm can be reliably fabricated to test their electrochemical performance and sensitivity for cortisol quantification. Cyclic voltammograms were collected with the LIG electrode of 2 mm in diameter show that the current increases with increasing scan rates from 20 to 100 mV/sec. The peak anodic current (Ipa) and peak cathodic current (Ipc) followed a linear relationship with the square root of the scan rate, suggesting a diffusion-limited voltammetry response.

Cyclic voltammograms were also collected with LIG electrodes of varying diameters to estimate the effect of miniaturizing the electrode on the electrochemical performance. The cyclic voltammetric response showed that the current increased with increasing electrode size. The maximum current was obtained for a 3 mm working electrode. The charge, determined by integrating the current with respect to potential, linearly increases with increasing the electrode area. This confirmed that the electrodes of varying diameters fabricated with the laser scribing approach provide a consistent electrochemical response. The Nyquist plots show that both real and imaginary magnitudes of impedance increased with decreasing electrode diameters. The electrochemical impedance magnitude and phase angle of the electrodes were recorded at a frequency range from 0.1 Hz to 100 kHz. The increased impedance with decreasing electrode size was consistent with previous reports. The LIG electrodes with 1 mm diameter were employed to construct sodium ion and cortisol sensors described below, as it only takes 2 μL sweat to fill the miniaturized sensing chamber.

### MIP Synthesis and Characterization

Pyrrole was employed as a monomer to synthesize MIP on LIG via electrochemical deposition in the presence of cortisol templates. After polypyrrole (PPy) deposition, the cortisol templates were removed from PPy to yield MIP for cortisol detection and quantification. The PPy without cortisol, i.e., non-imprinted polymer (NIP), serves as a control in evaluating the sensing performance of MIP-based cortisol sensors. The cyclic voltammetry curves corresponded to the 10th electrodeposition cycle of PPy using pyrrole in phosphate-buffered saline (PBS) with and without cortisol. The current was higher during the electrodeposition of the NIP than the MIP due to the presence of non-conducting cortisol inside the MIP matrix. SEM image reveals a thin layer of ˜100 nm PPy uniformly deposited on the graphene flakes. Representative Raman spectrum collected from PPy-coated LIG shows prominent Raman peaks at ˜1380 cm⁻¹ and 1570 cm^{-1,} which are convoluted with D and G Raman peaks from graphene. Peaks between 1380 cm⁻¹ and 1570 cm⁻¹ were attributed to the backbone C═C bonds stretching, C—N stretching, and inter-ring stretching C—C vibration mode of PPy. An asymmetric band at ˜1050 cm⁻¹ was attributed to C—H stretching vibration bands of polarons and bipolarons. Peaks at 975 cm⁻¹ and 935 cm⁻¹ corresponded to ring deformation associated with the polaron and bipolaron bands, indicating the oxidized form of PPy.

Following each step of the LIG electrode modification, the electrode impedance in PBS at frequencies ranging from 0.1 Hz to 10 kHz was recorded. The results showed that the PPy coatings significantly decreased the LIG electrode impedance magnitudes and phase angles due to the high conductivity of the oxidized PPy in the low-frequency range. At 0.1 Hz, the impedance of the PPy-coated electrode is ten-fold lower than the LIG electrode. The electrode with cortisol-embedded PPy exhibited higher impedance than the PPy without cortisol because cortisol hindered the flow of electrons and ions between the electrode and the surrounding electrolyte. After exposing the PPy-coated LIG electrodes to a mixture of acetic acid and methanol (7:3 v/v) to remove the cortisol templates, the electrochemical impedance further decreased due to the enhanced electrical conductivity of LIG electrodes following the chemical treatment with acetic acid. The impedance of NIP and MIP electrodes at 0.1 Hz decreased by half compared to that before exposing the electrodes to the template removal solution. The resultant MIP electrode showed a slightly higher impedance than the NIP. The higher impedance of the MIP electrode compared to the NIP electrode may result from two factors: the inherently higher resistance of the MIP electrode due to less deposition of PPy in the presence of cortisol and the residual cortisol templates left inside the MIP after the removal process. Considering wearable applications, the electrochemical impedance and electrical resistance of PPy-coated LIG electrodes was measured in a flat position and under bending. The impedance and resistance remain stable upon bending at a small radius of 2.4 cm, confirming that the devices can be applied to the forearm of pediatric and adult individuals and provide consistent measurements.

### MIP Cortisol Sensor Performance

Next, the sensitivity and specificity of MIP cortisol sensors was investigated, and the capability of monitoring cortisol in real time was demonstrated. A MIP cortisol sensor comprises three LIG electrodes, including one counter electrode, one Ag/AgCl-coated LIG as a reference electrode, and one MIP-LIG electrode as a working electrode. The devices with an NIP-LIG working electrode were also prepared for comparison. Using these three electrode devices, the impedance changes of MIP-LIG/NIP-LIG electrodes upon exposure to cortisol at the low-frequency range were analyzed. A MIP sensor was exposed to varying concentrations of cortisol from 0.1 pM to 1 μM in pH 6.5 artificial sweat, the electrochemical impedance was recorded at the frequency range from 0.1 to 10 Hz. The impedance increased with increasing concentrations of cortisol. The impedance increase induced by the captured cortisol at 0.1 pM concentration normalized with respect to the blank impedance (ΔZ/Z0) decreased with increasing frequency, following the same trend as the absolute impedance. The maximal impedance change (ΔZ/Z0) was obtained at 0.1 Hz, which was used for the following analysis. The dose-response curves collected from MIP and NIP devices after the devices were exposed to the cortisol solution were compared. The MIP device exhibited an increase in impedance with increasing cortisol concentrations, while the NIP device showed negligible changes. The MIP device can detect cortisol at concentrations from 0.1 pM to 1 μM. The plasma treatment was applied to modify the microfluidic surface to become hydrophilic, thereby facilitating sweat transport to the sensing chamber. However, this treatment may also alter the interaction between cortisol and the MIP. To assess the effect of plasma treatment on sensitivity, impedance measurements were conducted using the oxygen plasma-treated devices. After the plasma treatment, the overall impedance changes measured by the MIP device decreased while the linearity of impedance changes as a function of cortisol concentrations improved. The impedance changes in the NIP device remained negligible. The plasma-treated MIP device can detect and quantify cortisol at a low concentration of 1 pM, 1000-fold lower than previously reported MIP-based electrochemical sensors. These results confirmed that the MIP electrochemical sensor can provide highly sensitive cortisol quantification with electrochemical impedance measurements. The strong binding of cortisol to the MIP cavity is primarily due to a combination of hydrogen bonding and hydrophobic interactions. Although cortisol is uncharged, its binding alters the ion distribution on the charged PPy surface within the Debye length, resulting in impedance changes that enable the highly sensitive detection of cortisol. In these conductive MIP-based electrochemical sensors, the binding events occur within the Debye length, which is less than 1 nm under physiological conditions. This overcomes the charge screening limitations in electrochemical sensors that rely on nonconductive biorecognition elements.

To validate the selectivity of the MIP cortisol sensor, the sensor was exposed to several interfering molecules at physiologically relevant concentrations, including progesterone (200 pM), serotonin (10 nM), and cortisone (25 nM). Among these, cortisone and cortisol have similar chemical structures. The impedance changes from the interfering molecules remain below 7% of changes from 10 nM cortisol, confirming the high selectivity of the MIP cortisol sensor. To evaluate the real-time cortisol quantification, the MIP sensor in a microfluidic device was constructed, and concentrations of cortisol were changed from 0 pM to 100 nM sequentially. The electrochemical impedance was continuously recorded with a time interval of 27 seconds. A rapid increase in the impedance in the first 2 minutes after introducing a different concentration of cortisol was observed, and the impedance reached a plateau in ˜3 minutes. The impedance changes are consistent with those in the calibration curve. These results demonstrate that the MIP sensor can continuously quantify the cortisol concentration in real time. It is important to note that the channel width of the paperfluidic pump can be optimized to control the sweat removal rate based on the sensor's equilibrium time. It takes ˜2 minutes to fill the sensing chamber of ˜2.4 μL if sweat is rapidly produced with iontophoresis and intense cycling. The complete removal of the sweat with the paperfluid pump with a 0.5 mm wide channel width takes 3 minutes, allowing the sensor to reach equilibrium. With this design, the sensor accuracy is not affected by varying sweat secretion rates. The continuous quantification here is limited to increasing concentrations of cortisol because refreshing the MIP surface in situ is challenging. Despite this limitation, the capability of monitoring the increasing cortisol levels could still provide valuable insights into periods of heightened stress, allowing individuals to identify triggers and implement stress-reduction techniques.

### Sweat Sodium Ion Concentration Quantification

Sweat sodium ion concentration can reflect electrolyte imbalance-associated medical conditions, such as hyponatremia, muscle cramps, and dehydration. The physiological concentration of sodium ions in sweat falls in the range between 10 and 90 mM. An Na+ selective membrane was incorporated on top of the PPy-modified LIG electrode to quantify the sodium ion concentration. The open circuit potential (OCP) of the electrode increased after exposing it to increasing Na+ concentrations from 10 to 160 mM. The sensitivity was calculated to be around 64.9 mV/decade, which is close to the theoretical value of 59 mV/decade according to the Nernstian equation. The pH stability of the Na+ ion sensor was confirmed by exposing it to artificial sweat with varying pH levels, ranging from pH 5 to pH 7. The pH of the artificial sweat was adjusted with hydrochloric acid and potassium hydroxide. These results also demonstrate the consistent response of the Na+ sensor after exposure to varying concentrations of common ions in sweat, including potassium, hydrogen, chloride, and hydroxide.

The wearable device's performance for quantifying the Na+ and cortisol concentrations in situ on a healthy subject was validated using a freshly prepared device each time. The subject cycled on a stationary bike for 10 minutes at different times of the day (9:30 a.m., 1:30 p.m., and 5:30 p.m.) to produce sweat. The average sweat rates were quantified to be ˜2 μL/min. The sweat Na+ concentration varied between 19 mM and 40 mM. This falls into the normal range for the sweat produced by healthy individuals. Many factors can contribute to the fluctuation in ion concentration, such as dietary and water intake and physical load. Cortisol levels at different times of the day were analyzed. The cortisol level in the morning was quantified to be 4 nM, ten times higher than that in the afternoon. The decreasing trend follows the human circadian rhythm of cortisol expression, in which cortisol levels rise at the beginning of the biological day and decline throughout the day.

### Conclusions

In summary, the MIP-based electrochemical impedance biosensor reported here does not rely on labels or redox probes to achieve sensitive and specific detection and quantification of molecular biomarkers in sweat. The wearable sweat sensor combined with a sweat induction module and paper microfluidics enables real-time, simultaneous quantification of multiple parameters, including sweat volume, rate, sodium ion, and cortisol concentrations. It was demonstrated that the MIP-functionalized LIG sensor can provide real-time cortisol quantification at a low concentration of 1 pM, 1000-fold lower than previously reported MIP-based electrochemical sensors. The ion-selective membrane functionalized LIG electrodes can be used to quantify sweat sodium concentration at physiologically relevant ranges via open circuit potential measurements. The paper microfluidics can quantify the sweat volume and secretion rate and also reset the sensing chamber to continuously analyze the generated sweat. Although the current study focuses on cortisol sensing as a proof-of-concept, the MIP-based electrochemical sensors can extend to real-time detection and quantification of other biochemicals of interest, such as protein biomarkers and therapeutic drugs. The simultaneous multiparameter quantification approach could facilitate the diagnosis and monitoring of medical conditions based on the concentration of these biochemical biomarkers.

### Experimental Section

### Materials

Pyrrole, cortisol, cortisone, serotonin, progesterone, potassium ferrocyanide (II), potassium ferricyanide (III), silver nitrate, iron chloride, sodium ionophore, sodium tetrakis[3,5-bis(trifluoromethyl)phenyl]borate, polyvinyl chloride and bis(2-ethylhexyl) sebacate was purchased from Sigma-Aldrich. 10X phosphate buffered saline (PBS) was purchased from Gibco. Carbachol, cellulose chromatography paper (Whatman #1 grade), polyvinyl butyral, sodium citrate dihydrate, sodium chloride, iron (III) chloride, potassium hydroxide, Acetic acid, methanol, and sodium hydroxide were purchased from Fisher Scientific. Artificial eccrine sweat (pH 4.5) was obtained from Biochemazone. The pH of the artificial sweat was adjusted to pH 5-7 using an aqueous solution of 10 mM potassium hydroxide. The screen-printed carbon electrode was procured from Zensor. Polyimide films of 150 μm thick were obtained from CS Hyde Company. Medical grade double-sided adhesive (2477P) was purchased from 3M. Type 1 deionized water (18.2 mΩ·cm) was produced by the Sartorius Arium Pro Ultrapure water system. The LIG electrodes were plasma treated with high intensity for 1 minute using a plasma cleaner PDC-001 from Harrick Plasma.

### Fabrication and Modification of the LIG Electrodes

A 70 W CO₂ laser cutter (LS1630, Boss Laser) was used to fabricate LIG electrodes. The optimized fabrication parameters for electrodes and interconnects are power 10% and speed 55 mm/sec. Electrode with varying diameters of 1, 2, and 3 mm were fabricated. The flexible wires were bonded to the devices using heat-curable silver ink (Creative Materials). The interconnects were encapsulated with Tegaderm Transparent Film (3M). Ag/AgCl reference electrodes were prepared by the electrodeposition of silver on LIG electrodes using a mixture of silver nitrate and sodium citrate dehydrate as precursors with cyclic voltammetry from-0.9 to 0.9 V with 100 mV/sec scan rate using Reference 620+ Potentiostat (Gamry Instruments). After the electrodeposition, silver-coated LIG electrodes were exposed to 0.1 M FeCl3 aqueous solution for 30 seconds to obtain Ag/AgCl reference electrodes. To stabilize the reference electrodes, a cocktail of 79.1 mg of polyvinyl butyral (PVB) and 50 mg of sodium chloride (NaCl) in 1 ml of methanol was added to the electrode surface and dried overnight.

The cortisol MIP was synthesized on the LIG using the electropolymerization method. The LIG electrodes were exposed to a precursor solution of 37.5 mM pyrrole and 5 mM cortisol in 1X PBS and subjected to 10 cyclic voltammetry cycles from −0.2 to 0.9 V with a scan rate of 50 mV/sec. The electrodes were immersed in a solution of acetic acid and methanol (7:3 v/v) for 30 minutes to extract cortisol templates, followed by thorough rinsing with deionized water. The NIP was synthesized using the same procedure as MIP, except that no cortisol was present in the polymerization solution.

The Na+ selective membrane was fabricated on a PPy-coated LIG electrode. The PPy-coated LIG electrode was prepared the same way as the MIP electrode except cortisol was not added to the precursor solution. A cocktail was first prepared by dissolving 2 mg of Na ionophore X, 1.1 mg sodium tetrakis [3,5 bis(trifluoromethyl) phenyl] borate, 66 mg polyvinyl chloride, and 130.9 mg bis(2-ethylhexyl) sebacate into 1320 μL of tetrahydrofuran. This cocktail was then drop-casted on the PPy-coated LIG electrode, followed by overnight drying. The electrode was subsequently conditioned in an aqueous solution of 100 mM sodium chloride overnight.

### Microfluidic Device Characterization

The functional layers highlighted in FIG. 1a were assembled to form microfluidic devices. A blue dye aqueous solution was added to the inlets, and the flow profile of the microfluidic device was captured by video recordings. The wet properties of polyimide films with LIG electrodes with and without plasma treatment were compared. After the dye solution filled the sensing chamber, water was introduced to the inlets to evaluate the refreshability of the sensing chamber using the paperfluidic pump. The wicking rate of the paper microfluidics was quantified by video recording the fluid propagation after adding excess water to the inlet of the paper microfluidic channel.

The sweat secretion rate and volume were calculated from the video recordings of the paperfluidic devices. A healthy human subject of 22 years old performed 30 minutes of cycling while wearing the paperfluidic device on the forearm. For sweat stimulation, anode hydrogels were prepared by mixing 2 wt % carbachol and 2.4 wt % agarose solution at 100° C. and then added to the LIG electrode surface, followed by cooling at room temperature for solidification. Cathode hydrogels were prepared by replacing carbachol with 1 wt % sodium chloride. After applying the device on the forearm of the human subject, a small current of 100 μA was applied through the sweat stimulation electrodes to the skin for 5 minutes to induce sweat. Following the stimulation, the sweat secretion rate was quantified from the video recordings.

### Material and Sensor Characterization

SEM images of LIG and PPy-coated LIG were collected with field effect-scanning electron microscopy (JEOL JSM-7500F). The Raman spectra of LIG and PPy-coated LIG were recorded using a 514 nm laser with a Renishaw inVia confocal Raman spectrometer. The electrochemical characterization of LIG electrodes with varying diameters was performed in 5 mM K₄Fe(CN)₆/K₃Fe(CN)₆ electrolyte. The electrochemical impedance of LIG electrodes was recorded with a three-electrode configuration at frequencies ranging from 0.1 to 10 kHz with an AC voltage of 10 mV using Reference 620+ Potentiostat (Gamry Instruments).

Impedimetric response of a MIP sensor to varying concentrations of cortisol from 0.1 pM to 1 μM in artificial sweat was recorded. The MIP sensor was exposed to interfering molecules in artificial sweat, including progesterone (200 pM), serotonin (10 nM), and cortisone (25 nM), to evaluate the selectivity. The open circuit potential of the sodium ISM-coated electrode was recorded after the sensor was exposed to varying concentrations of NaCl from 10 to 160 mM. For in vivo demonstration, a human subject wore a device to quantify the sweat sodium ion and cortisol concentration at different times of the day, including 9:30 a.m., 1:30 p.m., and 5:30 p.m.

Although various embodiments of the present disclosure have been illustrated in the accompanying Drawings and described in the foregoing Detailed Description, it will be understood that the present disclosure is not limited to the embodiments disclosed herein, but is capable of numerous rearrangements, modifications, and substitutions without departing from the spirit of the disclosure as set forth herein.

The term “substantially” is defined as largely but not necessarily wholly what is specified, as understood by a person of ordinary skill in the art. In any disclosed embodiment, the terms “substantially”, “approximately”, “generally”, and “about” may be substituted with “within [a percentage] of” what is specified, where the percentage includes 0.1, 1, 5, and 10 percent.

The foregoing outlines features of several embodiments so that those skilled in the art may better understand the aspects of the disclosure. Those skilled in the art should appreciate that they may readily use the disclosure as a basis for designing or modifying other processes and structures for carrying out the same purposes and/or achieving the same advantages of the embodiments introduced herein. Those skilled in the art should also realize that such equivalent constructions do not depart from the spirit and scope of the disclosure, and that they may make various changes, substitutions, and alterations herein without departing from the spirit and scope of the disclosure. The scope of the invention should be determined only by the language of the claims that follow. The term “comprising” within the claims is intended to mean “including at least” such that the recited listing of elements in a claim are an open group. The terms “a”, “an”, and other singular terms are intended to include the plural forms thereof unless specifically excluded.

Conditional language used herein, such as, among others, “can”, “might”, “may”, “e.g.”, and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment.

While the above detailed description has shown, described, and pointed out novel features as applied to various embodiments, it will be understood that various omissions, substitutions, and changes in the form and details of the devices or algorithms illustrated can be made without departing from the spirit of the disclosure. As will be recognized, the processes described herein can be embodied within a form that does not provide all of the features and benefits set forth herein, as some features can be used or practiced separately from others. The scope of protection is defined by the appended claims rather than by the foregoing description. All changes which come within the meaning and range of equivalency of the claims are to be embraced within their scope.

Although various embodiments of the method and apparatus of the present invention have been illustrated in the accompanying Drawings and described in the foregoing Detailed Description, it will be understood that the invention is not limited to the embodiments disclosed, but is capable of numerous rearrangements, modifications and substitutions without departing from the spirit of the invention as set forth herein.

What is claimed is:

1. A wearable biosensor for real-time quantification of a biomarker in a biofluid, the wearable biosensor comprising:
at least one functional module wherein the at least one functional module comprises:
an iontophoresis induction module;
a microfluidic layer,
a plurality of multimodal biosensors, and
a paperfluidic layer.

2. The wearable biosensor of claim 1, wherein the iontophoresis induction module comprises two LIG electrodes coated with hydrogels.

3. The wearable sensor of claim 1, wherein the two LIG electrodes are an anode and a cathode.

4. The wearable biosensor of claim 3, wherein the anode electrode comprises a carbachol-loaded hydrogel coating and is positioned to capture the biofluid.

5. The wearable biosensor of claim 3, wherein the cathode electrode comprises a sodium chloride hydrogel coating and is configured to complete an electrical circuit.

6. The wearable biosensor of claim 2, wherein the microfluidic layer comprises an inlet layer.

7. The wearable biosensor of claim 6, wherein the inlet layer is made of double-sided skin adhesive that is configured to form a mechanically robust interface between the skin of a user and the wearable biosensor.

8. The wearable biosensor of claim 6, wherein the inlet layer comprise two arc shaped openings to allow direct contact between the hydrogel and the skin of a user.

9. The wearable biosensor of claim 6, wherein the inlet layer comprises one circular opening fluidly coupled to the paperfluidic layer for biofluid volume and rate quantification.

10. The wearable biosensor of claim 6, wherein the inlet layer has three circular openings to collect the biofluid for quantifying pH, sodium ion (Na+) and cortisol concentrations.

11. The wearable biosensor of claim 1, comprising a paper wicking layer that connects to the paperfluidic layer of the wearable biosensor to efficiently remove accumulated biofluid after quantification is completed.

12. The wearable biosensor of claim 1, wherein the wearable biosensor is encapsulated with a skin adhesive to minimize biofluid evaporation and to protect the device from environmental contamination.

13. The wearable biosensor of claim 2, wherein LIG electrodes are fabricated on polyimide sheet.

14. The wearable biosensor of claim 1, wherein the measured biomarker is one of cortisol, a hormone, a drug, a metabolite, a protein, or a pathogen.

15. The wearable biosensor of claim 1, comprising a first LIG electrode configured as a counter electrode, a second LIG electrode comprising an Ag/AgCl coating, and a pair of working electrodes.

16. The wearable biosensor of claim 15, wherein a working electrode of the pair of working electrodes is an LIG electrode comprising an electrochemically synthesized cortisol-specific Molecularly Imprinted Polymer coating for cortisol detection and quantification.

17. The wearable biosensor of claim 15, wherein a working electrode of the pair of working electrodes is an LIG electrode comprising a polypyrrole and sodium ion-selective membrane to quantify sweat Na+ concentration.

18. The wearable biosensor of claim 11, wherein the paper wicking layer comprises chromatography paper.

19. The wearable biosensor of claim 1, wherein the biofluid is sweat.

20. The wearable biosensor of claim 1, wherein the biofluid is interstitial fluid.
